(19) United States
(12) Patent Application Publication
(10) Pub. No.: US 2017/0107501 A1
(43) Pub. Date: Apr. 20, 2017

(54) Deoxyribonuclease Enzymes

(71) Applicant: THERMOFISHER SCIENTIFIC BALTICS UAB, Carlsbad, CA (US)

(72) Inventors: Gediminas ALZBUTAS, Vilnius (LT); Arunas LAGUNAVICIUS, Vilnius (LT); Milda KANIUSAITE, Vilnius (LT)

(21) Appl. No.: 15/316,114

(22) PCT Filed: Jun. 2, 2015
(86) PCT No.: PCT/EP2015/062222

§ 371 Date: Dec. 2, 2016

(30) Foreign Application Priority Data
Jun. 2, 2014 (GB) 1409709.1

(51) Int. Cl. C12N 9/22

(52) U.S. Cl. CPC C12N 9/22; C12Y 301/21001

(57) ABSTRACT

Provided is a deoxyribonuclease comprising: (a) an amino acid sequence having at least 85% sequence identity with the sequence of a eukaryotic DNase I; and (b) an amino acid sequence capable of binding nucleic acid non-specifically comprising at least one helix-hairpin-helix motif.

### FIELD OF THE INVENTION

The present invention relates to non-naturally occurring compositions of halophylic DNases, as well as uses of them in different kits and applications, e.g. RNA synthesis, purification and analysis.

### BACKGROUND OF THE INVENTION

In molecular biology research deoxyribonuclease I (DNaseI) is used in several applications, such as removal of genomic DNA from cell lysates, removal of plasmid from in vitro transcribed RNA, nick translation and DNaseI footprinting. One of the main disadvantages of wild type bovine DNaseI limiting its application in molecular biology manipulations is its low resistance to ionic strength. For example, the use of DNAseI to degrade residual genomic DNA in crude cell lysates in RNA sample preparation workflow is often not possible or requires extremely high DNaseI concentrations. As an alternative one could perform DNaseI treatment of isolated RNA sample, but this step requires subsequent DNaseI inactivation/removal thereby introducing additional manipulation steps and increased hands-on time.

It is known that increased ionic strength suppresses DNA binding activity of the enzyme via decreased ionic interactions that result from decreased dielectric constant. Therefore various approaches were developed aiming to overcome negative effect of high ionic strength on DNaseI enzymatic activity, such as rational design mutagenesis resulting in increased affinity of the enzyme for DNA. One way of doing this is to introduce additional positively charged amino acid residues (Arg, Lys) onto the protein surface interacting with DNA. Such an approach was employed in developing hyperactive human DNaseI (EP 0910647) as well as bovine DNaseI (EP 2213741).

An alternative approach to rational design mutagenesis has been used with other enzymes in order to try to alter their properties. This approach involves the creation of chimeric enzymes by fusing the enzymes with non-specific DNA binding domains. A few non-specific DNA binding proteins have been successfully employed to increase affinity for DNA and consequently improve various properties of some DNA polymerases: Phage phi 29 DNA polymerase (US 20120190014), Taq DNA polymerase (US20060234227), Pfu DNA polymerase (U.S. Pat. No. 8,232,078, EP 1910534). However, proteins that make good fusion candidates for one class of DNA modifying enzymes do not necessarily produce desired result when fused with another class of enzymes. In particular, DNA polymerases have a mode of DNA binding and mechanism of catalysis that is essentially different from that of DNaseI; DNA polymerases sequentially add nucleotides one by one to extend the 3′ end of an oligonucleotide, while DNaseI, in contrast, is an endonuclease that cleaves the phosphodiester bond within a polynucleotide chain in a non-sequential manner. Due to these differences the knowledge derived from the successful generation of useful chimeric DNA polymerases cannot be directly applied to constructing chimeric DNaseI proteins having the same properties. Moreover, a large number of DNA binding domains and proteins are known from across all domains of life (Eukaryota, Bacteria, Archaea, as well as from viral proteins) and the current state of the art provides little guidance to assist the researcher to choose among these. For example, U.S. Pat. No. 8,535,925 suggests a DNase I polypeptide further comprising a heterologous sequence-non-specific double-stranded DNA binding domain, and suggests that this is selected from the group consisting of a DNA binding domain from a Maf proto-oncogene transcription factor, an Sso family DNA binding protein and a HMf transcription factor. However, no evidence is provided that these domains result in a DNaseI enzyme with improved or useful properties. Therefore there is a need in the industry for identification of DNA binding proteins or protein domains which, when fused with DNaseI or other non-specific deoxyribonucleases would generate enzymes with improved properties.

### SUMMARY OF THE INVENTION

In a first aspect the present invention provides a deoxyribonuclease comprising a DNase I amino acid sequence and an amino acid sequence comprising at least one helix-hairpin-helix motif.

In particular, according to the first aspect the present invention provides a deoxyribonuclease comprising: (a) an amino acid sequence having at least 85% sequence identity with a eukaryotic DNaseI sequence; and (b) an amino acid sequence capable of binding nucleic acid non-specifically comprising at least one helix-hairpin-helix motif.

The present inventors have identified amino acid sequences with non-specific DNA binding properties that, when fused to a DNase I enzyme amino acid sequence, improve the deoxyribonuclease activity of the enzyme, particularly at high salt concentrations. The improved DNaseI enzymes of present invention possess properties that are superior if compared with wild type DNaseI and have particular utility in molecular biology applications. They are especially useful in the process of removing DNA from RNA preparations.

Accordingly, in a second aspect the present invention provides methods using the deoxyribonucleases of the present invention in the digestion of single-stranded and double-stranded DNA. In particular, this aspect provides a method for removing DNA from a sample, the method comprising contacting the sample with the deoxyribonuclease of the invention as described herein under conditions that allow the deoxyribonuclease to digest the DNA. In particular, the conditions comprise from 50 mM to 4M NaCl.

In a third aspect the present invention provides further products comprising the deoxyribonuclease of the invention. In this aspect the present invention provides a composition comprising the deoxyribonuclease of the present invention and a buffer. Further provided are kits comprising the deoxyribonuclease of the present invention and a reaction buffer, for removing nucleic acid from RNA preparations.

### DESCRIPTION OF THE FIGURES

FIG. 1 shows the effect of NaCl concentration on activity of DNase from Thioalkalivibrio sp. K90mix variants: (A, B)—intact DNaseTA from Thioalkalivibrio sp. K90mix; (C,D)—DNaseTA ΔC deletion mutant; (E, F)—DNaseTA H134A mutant. Left panel (A, C, E) represents substrate digestion reactions where NaCl concentration in reaction buffer varies from 0 M to 1M in 0.1 M increments. Right panel (B, D, F) represents substrate digestion reactions where NaCl concentration in reaction buffer varies from 0 M to 4M in 0.4 M increments. Control reactions (without enzyme) using 0, 1 and 4 M NaCl are denoted by K,0, K,1 and K, 4, respectively. Substrate used in for DNase degradation reactions: 2 μg of pUC19 DNA cleaved by SmaI. Concentration of enzymes in reaction mixtures: 2.5 nM. Reactions were performed 10′ at 37° C. in 100 μl of the reaction mixture, having composition as follows: 10 mM Tris—HCl, pH 7.5; 10 mM MgCl2; 10 mM CaCl2. DNA ladder—Thermo Scientific ZipRuler Express DNA Ladder 2.

FIG. 2 shows the effect of NaCl concentration on activity of DNase fusions with ComEA domains. (A, B)—DNaseI; (C,D)—DNaseI fusion with ComEA domain from Thioalkalivibrio sp. K90mix; (E, F)—DNaseI fusion with ComEA domain from Bacillus subtilis. Left panel (A, C, E) represents substrate digestion reactions where NaCl concentration in reaction buffer varies from 0 M to 1M in 0.1 M increments. Right panel (B, D, F) represents substrate digestion reactions where NaCl concentration in reaction buffer varies from 0 M to 4M in 0.4 M increments. Control reactions (without enzyme) using 0, 1 and 4 M NaCl are denoted by K,0, K,1 and K, 4, respectively.

FIG. 3 shows the activity of DNaseI and its fusion with ComEA domain variants at different ionic strength. Evaluation was performed by analyzing digestion of fluorescently labeled DNA duplex (30 bp). Corresponding fluorescence curves show relative fluorescence units (RFU) against time in seconds (t,s).

FIG. 4 shows the efficiency of DNA removal by DNaseI and its fusion variants when DNA is digested directly on a column filter during RNA purification procedure. The upper picture represents quantitative evaluation of undigested DNA remaining in eluates. The lower picture represents RT-qPCR results obtained using the same eluates. “A′” denotes analysis of undiluted qPCR reaction sample, when reverse transcriptase was not used, “B′” denotes 10-fold corresponding dilution and “C′” denotes 100-fold corresponding dilution. “A” denotes analysis of undiluted RT-qPCR sample, “B” denotes 10-fold corresponding dilution and “C” denotes 100-fold corresponding dilution.

FIG. 5 shows the efficiency of template DNA removal after in vitro transcription reaction using DNaseI and its fusion variants. Transcription reactions were performed using TranscriptAid™ T7 High Yield Transcription Kit” (Thermo Fisher Scientific). As a template 1 μg control DNA from the kit was used as a template. After transcription reaction (2 h, 37° C. temp.), undiluted and 5× diluted samples were treated with varying amounts of DNases equivalent to 1, 2, 5 Kunitz units of bovine DNase. Remaining DNA was detected by qPCR. “K”—denotes a control sample, which was not treated with DNase. “A”—denotes cases, when sample was not diluted before treatment with DNase. “B”—denotes cases, when sample was diluted 5 folds before treatment with DNase.

### DETAILED DESCRIPTION OF THE INVENTION

As indicated above, the present invention provides a deoxyribonuclease comprising a DNase I amino acid sequence and an amino acid sequence comprising at least one helix-hairpin-helix motif. In particular, the deoxyribonuclease comprises (a) an amino acid sequence having at least 85% sequence identity with a eukaryotic DNaseI; and (b) an amino acid sequence capable of binding nucleic acid non-specifically comprising at least one helix-hairpin-helix motif.

The deoxyribonuclease of the present invention is an endonuclease that non-specifically cleaves single- and/or double-stranded DNA. In particular, the deoxyribonuclease may release dinucleotides, trinucleotides, and oligonucleotides with 5′-phosphate and 3′-OH groups.

The DNase I enzyme part of the deoxyribonuclease may be a eukaryotic DNAse I or a mutant of a eukaryotic DNase I. Many DNase I enzymes derived from eukaryotes are known in the art, e.g. bovine, murine, human, etc., and their structures are known. Accordingly, it is known how to make amino acid substitutions, or to remove or add amino acids (such as tags) from the sequence of these enzymes in order to create mutants that retain DNase I/deoxyribonuclease activity. Moreover, functional mutants of these sequences and synthetic DNase I enzymes are also described in the art (see for example EP 2213741 and WO 97/47751). Accordingly, the DNase I amino acid sequence of the deoxyribonuclease has at least 85%, at least 90%, at least 95%, or at least 98% sequence identity with a eukaryotic DNase I. Percentage sequence identity is determine in the normal way, i.e. by comparing the sequence of the amino acid sequence with the reference sequence having the specified sequence identification number when the two sequences are optimally aligned.

Preferably the eukaryotic DNase I is a bovine DNase I. An example of a wild-type bovine DNase I has the amino acid sequence SEQ ID NO: 2 and is encoded by the nucleic acid sequence SEQ ID NO: 1. Accordingly, in a preferred embodiment of the invention the amino acid sequence of part (a) may have at least 85%, at least 90%, at least 95% or at least 98% sequence identity with SEQ ID NO: 2.

In particular, the deoxyribonuclease of the present invention is a fusion protein (a chimeric protein) that comprises at least two amino acid sequences attached together in a manner that is not usually naturally occurring, i.e. the deoxyribonuclease comprises a DNase I amino acid sequence and a heterologous amino acid sequence. The deoxyribonuclease of the present invention may further comprises a tag, such as an affinity tag, e.g. a his tag, or 1 to 10 terminal amino acids such as the terminal amino acids of shown in SEQ ID NO: 15, SEQ ID NO: 22 or SEQ ID NO: 37. In one embodiment the deoxyribonuclease consists of the DNase I amino acid sequence, the heterologous sequence, and a tag, or consists of the DNaseI amino acid sequence and the heterologous sequence.

The heterologous amino acid sequence is capable of binding non-specifically to DNA, and enhances the binding ability of the DNase I enzyme to single-stranded DNA and/or double-stranded DNA, preferably to double-stranded DNA, especially at high salt concentrations. The ability of the amino acid sequence to bind nucleic acid non-specifically refers to an ability to bind DNA in a non-sequence dependent manner.

The amino acid sequence capable of binding nucleic acid non-specifically comprises at least one helix-hairpin-helix DNA-binding motif, i.e. the amino acid sequence forms the structural motif of a helix-hairpin-helix. Such motifs are known in the art and are described, for example, in Doherty et al., (Nucleic Acids Research, 1996; 24(13): 2488-2497). In particular, the amino acid sequences of the at least one helix-hairpin-helix motif can be selected from those specified in Table 1 given in Doherty et al., or sequence variants thereof that retain the helix-hairpin-helix structural motif.

The amino acid sequence capable of binding nucleic acid non-specifically may comprise at least two helix-hairpin-helix motifs. In particular, the amino acid sequence capable of binding nucleic acid non-specifically may comprise two helix-hairpin-helix motifs in tandem.

Particular helix-hairpin-helix motifs of the invention are those known from prokaryotes, especially bacteria. Preferably the amino acid sequence of the helix-hairpin-helix motif is, or is based on, one from a ComEA protein. In Bacillus ComEA is encoded by the comG operon, however, other ComEA and ComEA-like proteins are known in the art (Provvedi et al., Molecular Microbiology, 1999; 31(1): 271-280). ComEA and ComEA-like proteins may be those identified as ComEA-like based on the Superfamily 1.7.4 database (Wilson D et al, Nucleic Acids Research, 2009; 37(Database issue): D380-6). Amino acid sequences forming a helix-hairpin-helix DNA-binding motif can be found in ComEA proteins and ComEA-like proteins obtained from organism of a genus selected from Bacillus, Thioalkalivibrio or Halomonas. In particular, they may be obtained from the halophilic bacteria Thioalkalivibrio sp. (strain K90mix) (Muyzer et al., Standards in Genomic Sci., 2011; 5: 341-355; predicted protein sequence at Uniprot accession D3SGB1), or Halomonas sp. TD01 (Cai L et al., Microb. Cell Fact., 2011; 10:88; predicted protein sequence at accession F7SPZ3). Preferably the helix-hairpin-helix motif is from ComEA from Bacillus subtilis (Inamine and Dubnau, J. Bacteriol., 1995, 177(11): 3045-51).

The helix-hairpin-helix motif may have an amino acid sequence selected from the group consisting of SEQ ID NO: 29, SEQ ID NO: 30, SEQ ID NO: 31, SEQ ID NO: 32, SEQ ID NO: 38 and SEQ ID NO: 39. Preferably the amino acid sequence capable of binding nucleic acid non-specifically comprises at least two of these sequences. More preferably the amino acid sequence capable of binding nucleic acid non-specifically comprises a helix-hairpin-helix motif having SEQ ID NO: 29 and a helix-hairpin-helix motif having SEQ ID NO: 30, or comprises a helix-hairpin-helix motif having SEQ ID NO: 31 and a helix-hairpin-helix motif having SEQ ID NO: 32, or comprises a helix-hairpin-helix motif having SEQ ID NO: 38 and a helix-hairpin-helix motif having SEQ ID NO: 39.

Where the amino acid sequence capable of binding nucleic acid non-specifically comprises more than one helix-hairpin-helix motif, these are usually attached together with a joining amino acid sequence. The joining amino acid sequence is not particularly limited but may be between 4 and 20 amino acids in length, preferably between 4 and 12 amino acids in length. Preferably the sequence of the HhH motifs are those, or are based on those, found in naturally occurring proteins (particularly ComEA proteins as indicated above). Often in these naturally occurring proteins two or more HhH motifs are found together in sequence. Therefore, the joining amino acid sequence can be based on the joining sequence found between naturally occurring HhH motifs. More preferably the amino acid sequence capable of binding nucleic acid non-specifically may comprise SEQ ID NO: 33, SEQ ID NO: 34 or SEQ ID NO: 40.

The amino acid sequence having at least 85% sequence identity to SEQ ID NO: 2 and the amino acid sequence capable of binding nucleic acid non-specifically may form a fusion protein, with a linker amino acid sequence. In particular, the amino acid sequence capable of binding nucleic acid non-specifically may be attached to the C-terminal end or the N-terminal end of the amino acid sequence having at least 85% sequence identity to SEQ ID NO: 2. Preferably the amino acid sequence capable of binding nucleic acid non-specifically is linked to the C-terminal end of an amino acid sequence having at least 85% sequence identity with a eukaryotic DNaseI sequence.

The sequence of the linker is not especially limited. However, most preferably the linking sequence is based on the amino acid sequence of the ComEA protein immediately adjacent to the helix-hairpin-helix structure. In particular, as indicated above for the joining amino acid sequence, preferably the sequence of the HhH motifs are those, or are based on those, found in naturally occurring proteins (particularly ComEA proteins as indicated above). Therefore, the amino acid sequence that is found linking these HhH motifs to other domains of the naturally occurring protein can be used as basis for the linker sequence used in the present invention. However, it is preferred that the linker is between 15 and 35 amino acids in length, more preferably between 25 and 35 amino acids in length. The linker may be selected from SEQ ID NO: 42, SEQ ID NO: 43 or SEQ ID NO: 44. In a preferred embodiment the amino acid sequence capable of binding nucleic acid non-specifically includes a linker and the amino acid sequence comprises SEQ ID NO: 35, SEQ ID NO: 36 or SEQ ID NO: 41.

In relation to the amino acid sequence capable of binding nucleic acid non-specifically it will be appreciated that this can have at least 85%, at least 90%, at least 95% or at least 98% sequence identity with the sequences specified above, provided that the structural motif of the helix-hairpin-helix is retained. In particular, the sequences may include amino acids substitutions (particularly conservative substitutions), or addition or deletion of amino acids. Preferably the substitutions within the helix-hairpin-helix sequences identified above are conservative substitutions or substitutions based on other helix-hairpin-helix sequences known in the art. In a more preferred embodiment the specified sequences of the helix-hairpin-helix sequences is retained while the variation is within the joining amino acid sequence between the helix-hairpin-helix sequences and the linker amino acid sequences.

Therefore in one embodiment the present invention provides a deoxyribonuclease comprising (a) an amino acid sequence having at least 85% to SEQ ID NO: 2; and (b) an amino acid sequence capable of binding nucleic acid non-specifically and consisting of:
(i) a sequence having at least 85% sequence identity with SEQ ID NO: 15, wherein the sequence having at least 85% sequence identity with SEQ ID NO: 15 comprises SEQ ID NO: 31 and SEQ ID NO: 32;
(ii) a sequence having at least 85% sequence identity with SEQ ID NO: 22, wherein the sequence having at least 85% sequence identity with SEQ ID NO: 22 comprises SEQ ID NO: 29 and SEQ ID NO: 30; or
(iii) a sequence having at least 85% sequence identity with SEQ ID NO: 37, wherein the sequence having at least 85% sequence identity with SEQ ID NO: 37 comprises SEQ ID NO: 38 and SEQ ID NO: 39.

In a preferred embodiment of the present invention provides a deoxyribonuclease consisting of (a) an amino acid sequence having at least 85% sequence identity with a eukaryotic DNaseI, preferably having 85% sequence identity to SEQ ID NO: 2; and (b) SEQ ID NO: 15, SEQ ID NO: 22 or SEQ ID NO: 37.

In a further preferred embodiment the present invention provides a deoxyribonuclease of SEQ ID NO: 24 or SEQ ID NO: 26.

As indicated above, the deoxyribonucleotides of the present invention have properties that are advantageous compared to wild-type DNaseI, preferably wherein the wild-type DNaseI is one having SEQ ID NO: 2. The amino acid sequence capable of binding nucleic acid non-specifically (the heterologous amino acid sequence) enhances the ability of the deoxyribonuclease to bind DNA.

In particular, the heterologous amino acid sequence enhances the ability of the DNase I enzyme to bind to DNA at high salt concentrations. Accordingly, the deoxyribonuclease of the present invention has a higher activity at elevated NaCl concentrations (in particular at values above 100 mM, and preferably in the range of 50 mM to 4M, most preferably in the range of 50 to 200 mM) than a wild-type bovine DNase I enzyme having SEQ ID NO:2 (and encoded by SEQ ID NO:1).

Therefore, the deoxyribonuclease according to the present invention may have an activity at 50 Mm NaCl, 100 mM NaCl, 200 mM NaCl, and/or 1M NaCl that is greater than that of a wild-type bovine DNase I having SEQ ID NO: 2. This activity may be assessed using the methods for measuring DNaseI activity known in the art (such as those used in the Examples herein) and in particular using conditions (other than salt concentration) that are otherwise known to be the optimum conditions for the wild-type DNaseI activity.

The deoxyribonuclease according to the present invention may have a higher affinity to double-stranded and/or single-stranded DNA compared to a wild-type bovine DNaseI having SEQ ID NO: 2. The deoxyribonuclease may also have a higher processivity compared to wild-type bovine DNase I having SEQ ID NO: 2.

The deoxyribonuclease described herein may be considered as an isolated deoxyribonuclease. By “isolated” it is meant that the deoxyribonuclease is separated from the components, e.g. cells, with which it may potentially be found in nature.

The deoxyribonuclease of the present invention may be comprised in a composition which also comprises a buffer. The buffer may be a storage buffer, in which the deoxyribonuclease can be stored and transported. The buffer may comprise at least one of TrisHCl, CaCl₂, MgCl₂ and glycerol. The buffer may be a reaction buffer and may comprise at least one of TrisHCl, CaCl₂, MgCl₂, or MnCl₂.

The present invention also provides a kit comprising a deoxyribonuclease according to the invention and a reaction buffer. In particular, the deoxyribonuclease and the reaction buffer are separately packaged. The deoxyribonuclease may be comprised in a composition which also comprises storage buffer as indicated above. The reaction buffer may be as indicated above. The kit may further comprise instructions for use of the kit. In particular, the kit may be suitable for: (i) preparation of DNA-free RNA, (ii) removal of template DNA following in vitro transcription, (iii) preparation of DNA-free RNA prior to RT-PCR and RT-qPCR, (iv) DNA labeling by nick-translation in conjunction with DNA Polymerase I, (v) studies of DNA-protein interactions by DNase I, RNase-free footprinting, or (vi) generation of a library of randomly overlapping DNA inserts.

In a further aspect the present invention provides the use of a deoxyribonuclease according to the present invention to digest DNA in a sample. Similarly, a method is also provided for removing DNA from a sample comprising contacting the sample with the deoxyribonuclease of the invention under conditions that allow the deoxyribonuclease to digest the DNA. In particular, the sample may comprise RNA.

As indicated above, the deoxyribonuclease of the present invention has higher resistance to ionic strength as compared to wild-type DNaseI, and in particular as compared to a wild-type bovine DNase I having SEQ ID NO: 2. As such, the deoxyribonuclease of the present invention may be efficiently utilized at higher salt concentrations than those utilized with the wild-type DNaseI. Accordingly, in the method of the present invention the conditions that allow the deoxyribonuclease to digest the DNA may include a concentration of from 50 mM to 4 M NaCl, more preferably from 50 to 2M NaCl or from 50 to 200 mM NaCl.

Moreover, the deoxyribonuclease of the present invention may be efficiently utilized at lower enzyme concentrations than that required with wild-type DNaseI. The deoxyribonuclease of the present invention has particular utility in the following: (i) preparation of DNA-free RNA, (ii) removal of template DNA following in vitro transcription, (iii) preparation of DNA-free RNA prior to RT-PCR and RT-qPCR, (iv) DNA labeling by nick-translation in conjunction with DNA Polymerase I, (v) studies of DNA-protein interactions by DNase I, RNase-free footprinting, or (vi) generation of a library of randomly overlapping DNA inserts.

In a further aspect the present invention further provides a polynucleotide (or nucleic acid sequence) encoding the deoxyribonuclease according to the present invention. The polynucleotide can comprise deoxyribonucleotides or ribonucleotides. In particular, the polynucleotide encoding the deoxyribonuclease according to the present invention comprises SEQ ID No: 23 or SEQ ID NO: 25, or a sequence having at least 85% sequence identity thereto. In particular, the sequence having at least 85% sequence identity thereto may comprise substitution mutations of SEQ ID NO: 23 or SEQ ID NO: 25 based on codon degeneracy.

The polynucleotide may be comprised in a vector. Preferably the vector is one which can be used for the replication and optionally also the expression of the polynucleotide. Suitable vectors are known in the art, but may be plasmids, such as those utilized in the Examples of the present application. In particular, the vector is an expression vector. In the expression vector the polynucleotide may be operably linked to a control sequence, such as a promoter or enhancer sequence, which controls the expression of the polynucleotide.

The present invention also provides a host cell which comprises the vector or the polynucleotide of the present invention as described herein. The host cell may be prokaryotic or eukaryotic. In one embodiment the host cell is a bacterial cell.

The polynucleotide, vector or host cells described herein can be used in a method of producing the deoxyribonuclease of the invention. In particular, in a further aspect the present invention provides a method of making the deoxyribonuclease comprising the steps of culturing the host cell under conditions which allow for the expression of the deoxyribonuclease. Suitable conditions for producing DNaseI are known in the art and may be utilized to produce the deoxyribonuclease of the present invention.

The invention will now be described in further detail, by way of example only, with reference to the following Experiments and related Figures.

### EXAMPLES

### Abbreviations

DNaseTA—DNase from Thioalkalivibrio sp. K90mix.

ComEA—Competence protein ComEA, helix-hairpin-helix domain (IPR004509).

DNaseBS—DNaseI fusion with ComEA domain from Bacillus subtilis.

DNaseDT—DNaseI fusion with ComEA-like domain from Thioalkalivibrio sp. K90mix.

DNaseI—bovine DNaseI.

RT-qPCR—real-time reverse-transcription polymerase chain reaction

### Example 1

Properties of the predicted protein of UniProt Accession No. D3SGB2 (putative DNase from Thioalkalivibrio sp. K90mix) were investigated by producing and analyzing recombinant version of this protein (designated DNaseTA) expressed in E. coli. Mutants of DNaseTA were made (DNAseTA AC and DNAse TA H134A) and tested.

### Example 1.1 Cloning and Expression of DNase from Thioalkalivibrio sp. K90Mix

Gene sequence (without secretion signal) encoding putative DNase from Thioalkalivibrio sp. K90mix (accession D3SGB1) was optimized using DNA 2.0 package for expression in E. coli, synthesized and cloned into pLATE31 expression vector using aLICator™ LIC Cloning and Expression Kit 3 (Thermo Fisher Scientific). Coding nucleotide sequence is presented in SEQ ID NO: 3.

For protein expression E. coli ER2566 strain was transformed with pLATE51 vector carrying cloned gene for DNase DT. Bacteria were grown in LB broth supplemented with glucose (1%) and carbenicillin (100 μg/ml). Initially a pre-culture was prepared for inoculation up to ˜0.3 OD₆₀₀, main culture was inoculated with 1/40 of the pre-culture. Induction of expression was performed by addition of IPTG (up to 1 mM) when OD₆₀₀ reached ˜0.8-0.9. Before induction the culture was cooled on ice and after induction bacteria were grown at 23° C. for 16 h. Pre-culture and main culture before induction were incubated at 37° C.

One step purification of expressed protein was performed using nickel affinity chromatography with spin columns equivalent to HisPur Ni-NTA Spin Columns (Thermo Fisher Scientific).

### Example 1.2 Construction of DNaseTA H134A and DNaseTA ΔC Deletion Mutants

Creating H134A mutant of DNaseTA two step megaprimer PCR was employed. Both PCR reactions were performed with 2× Phusion® High-Fidelity PCR Master Mix (Thermo Fisher Scientific). The first PCR was performed using primers, which sequences are given in SEQ ID NO: 5 and SEQ ID NO: 6 as a template DNaseTA plasmid DNA (cloned as described in Example 1.1) was used. The PCR product was gel-purified and used for the second PCR reaction. This PCR product was used in the second PCR reaction together with a primer, which sequence is given in SEQ ID NO: 7. As a template the plasmid of DNaseTA was used (see above). The resulting fragment was gel-purified and cloned to pLATE31 vector using aLICator™ LIC Cloning and Expression Kit 2 (Thermo Fisher Scientific). For cloning and plasmid purification Escherichia coli strain ER2267 was used. The coding sequence and amino acid sequence of DNaseTA inactive site mutant are given in SEQ ID NO: 8 and SEQ ID NO: 9.

For creation of DNaseTA ΔC deletion mutant one step PCR was employed. PCR reaction was performed with Phusion® High-Fidelity PCR Master Mix (Thermo Fisher Scientific). Primers which sequences are given as SEQ ID NO: 10 and SEQ ID NO: 11 were used. As a template DNaseTA plasmid DNA (cloned as described in Example 1.1) was used. The resulting fragment was gel-purified and cloned to pLATE31 vector using aLICator™ LIC Cloning and Expression Kit 2 (Thermo Fisher Scientific). For cloning and plasmid purification Escherichia coli strain ER2267 was used.

The coding sequence and amino acid sequence of DNaseTA ΔC deletion mutant without C-terminal domain are given in SEQ ID NO: 12 and SEQ ID NO: 13.

Expression of both mutants was performed identically. pLATE31 vectors with the cloned proteins were transformed to E. coli ER2566 strain. Bacteria were grown in LB broth supplemented with glucose (1%) and carbenicillin (100 μg/ml). Initially a preculture was grown till OD₆₀₀ reached ˜0.3. Main culture was inoculated with 1/40 of the pre-culture. Induction of expression was performed by addition of IPTG (up to 1 mM) when OD₆₀₀ reached ˜0.8-0.9. Before induction the culture was cooled on ice and after induction bacteria was grown at 23° C. 16 h. Pre-culture and main culture before induction were incubated at 37° C.

### Example 1.3. Evaluation of Activity of DNaseTA and its Mutants DNAseTAAC and DNAseTAH134A

10 nM 16 bp DNA (2 nM were labeled with ³³P at 5′) was used as a substrate. Reaction mixtures contained 0.66 nm of the enzyme or its mutants. DNA digestion was performed at 37° C. in 100 μl reaction buffer (10 mM Tris-HCl, pH7.5; 10 mM CaCl2; 10 mM MgCl2). 9 μl samples of reaction mixes were taken out at 1, 2, 4, 8, 16, 32, 64, 128, 192 minutes after start and mixed with 9 μl of 2×RNA loading dye (Thermo Fisher Scientific). These mixes were heated for 5′ at 95° C. and analyzed by denaturing PAGE. Halve times of substrate digestion were estimated in comparison with undigested substrate band (control) using densitometry analysis.

Results

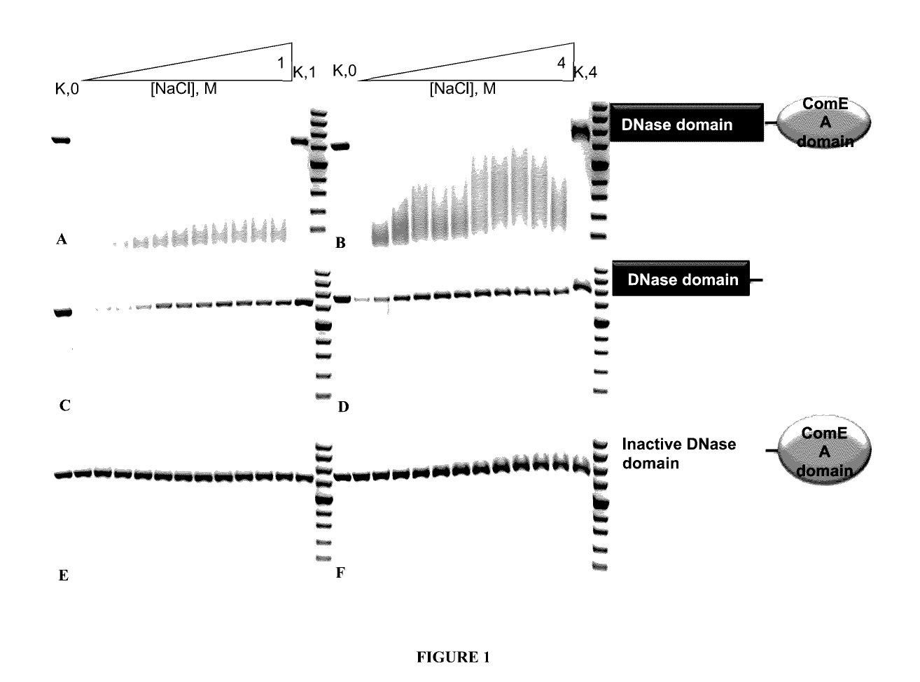

Obtained recombinant enzyme was designated as DNaseTA and its ability to catalyze DNA degradation in high ionic strength (salt) conditions was evaluated. As it is seen in FIG. 1, DNaseTA remains active even at high ionic strength. Moreover, this DNase digests DNA at 4 M NaCl concentration even better than at 3.2 M NaCl concentration. Our data confirm that putative DNaseTA is an active DNase and is extremely salt tolerant. DNaseTA is composed from two domains: an N-terminal DNase domain and, based on Superfamily 1.7.4 Database (Wilson D et al, 2009), a C-terminal ComEA-like domain.

To test whether ComEA-like domain from DNaseTA is a key factor in determining salt tolerance we have constructed two DNaseTA mutants: (a) a truncated protein without a C-terminal ComEA-like domain (designated DNaseTA ΔC), and (b) a mutant—with a mutation in the active site of DNase domain, where a catalytic histidine was changed to alanine (designated DNaseTA H134A). We have assayed the ability of both mutants to degrade substrate DNA in the presence of increasing NaCl concentrations. As seen in FIG. 1 (C, D, E, F), the enzyme lacking ComEA-like domain still retains DNase activity, although its salt tolerance is decreased. On the other hand, the mutant that harbors inactivating mutation at the DNase active site is totally inactive. These data imply that ComEA-like domain has no effect on DNase activity and wild type DNaseTA employs the same active site for DNA digestion both in low ionic strength and in high ionic strength conditions, while ComEA-like domain is responsible for salt tolerance in DNaseTA.

In addition, we have assayed activity of DNaseTA and its mutants by observing digestion of radioactively labeled substrate in buffers of different ionic strength. Results are presented in Table 1.

TABLE 1
Radioactive substrate digestion half-life for wild type DNase from
Thioalkalivibrio sp. K90mix (DNaseTA) and its mutants.

| | DNaseTA | DNaseTA ΔC | DNaseTA H134A |
|---|---|---|---|
| NaCl, M | Half-life of substrate digestion T_{1/2}, min. | | |
| 0 | 9.43 | 31.29 | ND |
| 0.5 | 43.39 | ND | ND |
| 1.2 | 60.18 | ND | ND |
| 4.0 | 47.66 | ND | ND |

ND—not detectable

Results, presented in Table 1 confirm data presented in FIG. 1. DNaseTA substrate digestion half time increases only ˜5 fold when concentration of NaCl increases from 0 M to 0.5 M and further to 4M of NaCl. Noteworthy, in the range of NaCl concentrations from 0.5 M NaCl to 4 mM NaCl DNaseTA substrate digestion half-life essentially remains the same despite increasing ionic strength. On the opposite, DNaseTA ΔC mutant that has no ComEA-like domain is inactivated by ionic strength corresponding to 0.5 m NaCl, while the mutant DNaseTA H134A is inactive at any ionic strength.

### Example 2

In view of the above, we tested ComEA-like domain from DNaseTA as potential fusion partner for bovine DNaseI. Protein sequence identity between DNase domains of bovine DNaseI and DNase TA is only ˜27% (sequences are given as SEQ ID NO: 2 and SEQ ID NO: 4, respectively), therefore the likelihood of success, namely, that ComEA-like domain of bacterial DNaseTA would enhance salt tolerance for bovine DNaseI is not obvious and requires experimental verification. So far there are no known protein sequences where ComEA-like domain would be naturally associated with any eukaryotic DNase.

ComEA-like domain from Thioalkalivibrio sp. K90mix chosen as candidate for fusion with DNaseI is not characterized experimentally; there is no available information about its DNA binding properties in scientific literature. Other possible imperfection of this ComEA-like domain as potential fusion partner is that the source organism Thioalkalivibrio sp. K90mix, where this domain was identified is classified as an extreme halophile and, most probably, this domain is well adapted for extremely high ionic strength conditions and may have limiting effect on DNaseI activity at lower ionic strength conditions. However many applications where DNaseI is routinely used (e.g. RT-qPCR) utilize buffers of relatively low salt concentrations. Therefore it would be rational to test several candidate fusion partners with bovine DNaseI, which come from microorganism proliferating at differing salt conditions.

We chose ComEA domain from Bacillus subtilis (Inamine GS and Dubnau D, 1995) as the second fusion partner for bovine DNaseI. This domain has non sequence specific DNA binding and specificity for dsDNA (Provvedi R and Dubnau D, 1999). It is known that not all ComEA-like domains are specific for dsDNA (Jeon B and Zhang Q, 2007). It was hoped that DNaseI fusion with ComEA domain from Bacillus subtilis should result in specific increase of chimeric protein activity on dsDNA substrate in buffers with higher ionic strength without any change of activity on ssDNA substrate as ComEA domain would compensate decreased DNA binding at higher ionic strength characteristic for DNaseI.

Following bioinformatics analysis, two potential ComEA-like domains were selected for experiments as candidate partners for C-terminal fusion with DNaseI:
ComEA-like domain from Thioalkalivibrio sp. K90 mix. This domain is naturally found in multidomain protein having Uniprot accession number D3SGB1.
ComEA domain from Bacillus subtilis. This domain is naturally found in multidomain protein having Uniprot accession number P39694.

Linkers were selected based on bioinformatics analysis of those naturally found in multidomain proteins being considered.

The ComEA-like domain from Thioalkalivibrio sp. K90mix is naturally found in DNaseTA enzyme comprising DNase and ComEA-like domains. Therefore the natural linker existing between these domains was used when fusing ComEA-like domain with bovine DNaseI.

The ComEA domain from Bacillus subtilis, which was used as the second fusion candidate, comes from ComE operon protein 1 (Uniprot accesion P39694). This protein is also multidomain and it is essentially composed from three parts: a membrane anchor, linker and a DNA receptor. Therefore when fusing ComEA domain with bovine DNaseI we selected the linker based on the natural linker.

### Example 2.1. Construction of Mammalian DNaseI Fusions with ComEA-Like Domains

ComEA-like domain from Thioalkalivibrio sp. K90mix was fused to C terminus of bovine DNaseI. The resulting chimeric protein was designated “DNaseDT”. Nucleotide and amino acid sequences of the chimeric protein are presented as SEQ ID NO: 25 and SEQ ID NO: 26, respectively.
ComEA domain from ComE operon protein 1 (Uniprot code P39694) from Bacillus subtilis was fused to C terminus of bovine DNaseI. The resulting chimeric protein was designated “DNaseBS”. Nucleotide and amino acid sequences of the chimeric protein are presented as SEQ ID NO: 23 and SEQ ID NO: 24, respectively.

Coding nucleotide and corresponding amino acid sequences for ComEA-like domain (including the linker sequence) from Thioalkalivibrio sp. K90mix are given as SEQ ID NO: 14 and SEQ ID NO: 15. Coding nucleotide and corresponding amino acid sequences for ComEA domain from ComE operon protein 1 (including the linker sequence) are given as SEQ ID NO: 21 and SEQ ID NO: 22.

The DNaseDT fusion was constructed and cloned into pLATE51 vector as follows:
Two step megaprimer PCR was employed. Both PCR reactions were performed with 2× Phusion® High-Fidelity PCR Master Mix (Thermo Fisher Scientific). The first PCR was performed using primers, which sequences are given as SEQ ID NO: 16 and SEQ ID NO: 17, using pLATE31 plasmid with cloned DNaseTA gene as a template. The PCR product was gel-purified and used for the second PCR reaction together with a primer, which sequence is given as SEQ ID NO: 18, and plasmid carrying cloned bovine DNaseI gene (SEQ ID No: 1) as a template. The resulting fragment was gel-purified and cloned to pLATE51 vector using aLICator™ LIC Cloning and Expression Kit 2 (Thermo Fisher Scientific). Escherichia coli strain ER2267 was used for cloning and plasmid analysis.
The DNaseBS fusion was constructed and cloned to pLATE51 vector as follows:
Two step megaprimer PCR was employed. Both PCR reactions were performed with 2× Phusion® High-Fidelity PCR Master Mix (Thermo Fisher Scientific). The first PCR was performed using primers, which sequences are given in SEQ ID NO: 19 and SEQ ID NO: 20 using Bacillus subtilis genomic DNA as a template. The PCR product was gel-purified and used for the second PCR reaction together with a primer, which sequence is given as SEQ ID NO: 18, and plasmid carrying cloned bovine DNaseI gene (SEQ ID No: 1) as a template. The resulting fragment was gel-purified and cloned to pLATE51 vector using aLICator™ LIC Cloning and Expression Kit 2 (Thermo Fisher Scientific). Escherichia coli strain ER2267 was used for cloning and plasmid analysis.
For protein expression pLATE51 vectors with the cloned chimeric DNases were transformed to E. coli ER2566 strain. Bacteria were grown in LB broth supplemented with glucose (1%) and carbenicillin (100 μg/ml). Initially a pre-culture was grown for inoculation up to ˜0.3 OD₆₀₀, main culture was inoculated with 1/40 of the pre-culture. Induction of expression was performed by addition of IPTG (up to 1 mM) when OD₆₀₀ reached ˜0.8-0.9. Before induction the culture was cooled on ice and after induction bacteria was grown at 23° C. for 16 h. Pre-culture and main culture before induction were incubated at 37° C.
One step purification of the fusion proteins was performed using nickel affinity chromatography with spin columns equivalent to HisPur Ni-NTA Spin Columns (Thermo Fisher Scientific).
Along with the fusion proteins wild type bovine DNaseI and DNaseTA were purified following identical procedures.

### Example 2.2 Digestion of DNA Substrates with Chimeric DNases in Buffers of Different Ionic Strength

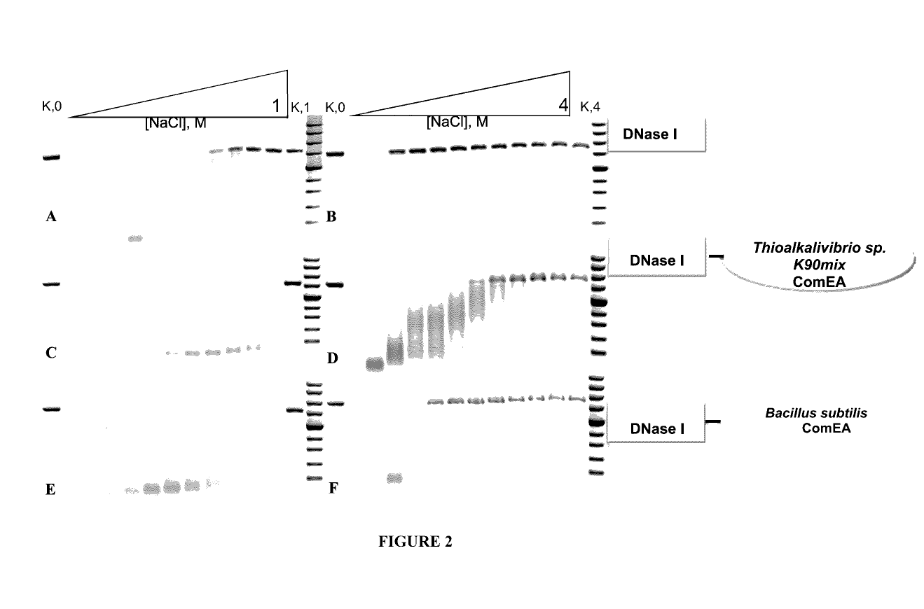

Long DNA substrate (2686 bp) was digested with DNases of present invention in buffers containing variable amounts of sodium chloride. Results are presented in FIG. 2.

For each enzyme two series of experiments were performed: in the first case NaCl concentration varied from 0 M to 1M in 0.1 M increments, while in the second case NaCl concentration varied from 0 M to 4M in 0.4 M increments. The substrate used was 2 μg pUC19 DNA cleaved with SmaI. Concentrations of enzymes were 2.5 nM per reaction. Reactions were performed for 10′ at 37° C. in 100 μl of the reaction mixture, which composition was as follows: 10 mM Tris—HCl, pH 7.5; 2.5 mM MgCl2; 0.1 mM CaCl2. Thermo Scientific ZipRuler Express DNA Ladder 2 was used as molecular weight standard to evaluate DNA substrate degradation.

Additionally we have evaluated digestion of short DNA substrate by the chimeric DNases of present invention by observing radioactively labeled substrate digestion in buffers of different ionic strength. As a substrate we used 10 nM 16 bp DNR (2 nM were labeled with ³³P at 5′-end). Reaction mixtures contained 0.66 nm of the enzymes. DNA digestion was performed at 37° C. in 100 μl reaction buffer (10 mM Tris—HCl, pH 7.5; 10 mM CaCl2; 10 mM MgCl2). 9 μl samples of reaction mixes were taken out at 1, 2, 4, 8, 16, 32, 64, 128, 192 minutes after start and mixed with 9 μl of 2×RNA loading dye (Thermo Fisher Scientific). These mixes were heated for 5′ at 95° C. and analyzed by denaturing PAGE. From the densitometric analysis of undigested substrate band the halve times of substrate digestion were estimated and results are presented in Table 2.

### Example 2.3 Digestion of Fluorescently Labeled DNA Duplex

Further evaluation of activity of DNaseI and its fusion with ComEA domain variants at different ionic strength was performed by analyzing digestion of fluorescently labeled DNA duplex (30 bp). After nuclease enzyme is added to reaction mix substrate degradation begins, flourophore is released. As a substrate a dual labeled duplex was used, which was produced by hybridizing the following single stranded oligonucleotides: 5′-GTTGGTGGGTTTGGGTGTGGGTTTGTGTTT-BHQ1-3′ (SEQ ID NO: 27) and 5′-FAM-AAACACAAACCCACACCCAAACCCACCAAC-3′ (SEQ ID NO: 28). Reactions were prepared in the following buffer: 10 mM Tris—HCl, 3 mM EDTA, 1% Triton X-100, 1 mg/ml BSA. 0.2 μM of DNA duplex and 0.044 nM concentrations of relevant DNase enzyme (DNaseI, DNaseBS or DNaseDT) were used. Reactions were started by addition of 10× start solution containing 40 mM CaCl₂ and 100 mM Mg acetate. The fluorescence was monitored in 12 seconds intervals and for each curve a maximum fluorescence change rate was calculated, which should be proportional to enzymatic activity. Such analyzes were performed by varying amounts of NaCl in the final reaction buffer in order to estimate activity decrease due to ionic change

Results

Both DNaseDT and DNaseBS were cloned and expressed in E. coli as described above. It is known that when expressed in E. coli bovine DNaseI is extremely toxic for host cells and special techniques are necessary to obtain sufficient yields of recombinant DNaseI protein. Noteworthy, both fusions (DNaseDT and DNaseBS) were even more toxic to E. coli host cells. Even though the yields of both fusion proteins were lower as compared to that of recombinant bovine DNaseI, we were able to collect sufficient amounts of soluble DNaseDT and DNaseBS proteins for further analysis.

ComEA Type Domains Enhance DNaseI Activity in Buffers Containing Salt

Certain molecular biology techniques require DNA digestion to be performed in extremely high ionic strength conditions, like degradation of contaminating gDNA in purified RNA samples, while keeping RNases inactive by high salt concentration.

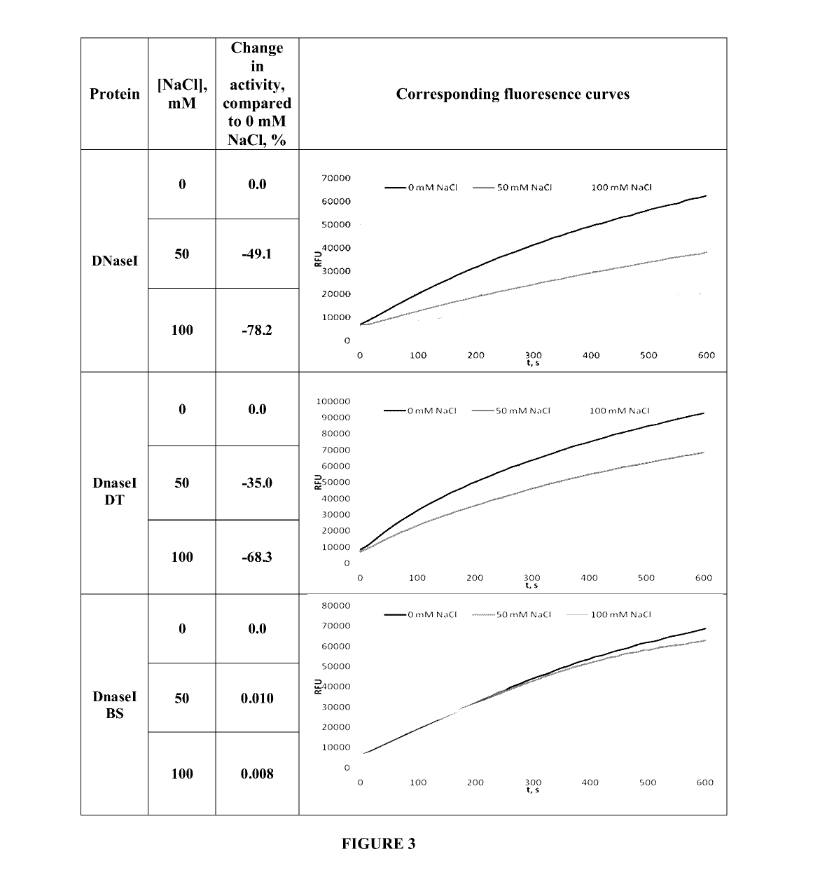

To test salt tolerance of both DNase fusions we have assayed activity of DNaseDT and DNaseBS by observing digestion of radioactively labeled substrate in buffers of different ionic strength. Results are presented in Table 2 and FIGS. 2 and 3.

TABLE 2
Radioactive substrate digestion half-life for wild type bovine DNaseI and
chimeric proteins DNaseDT and DNaseBS.

| | DNaseI | DNaseDT | DNaseBS |
|---|---|---|---|
| | Half-life of substrate | | |
| NaCl, M | digestion T_{1/2}, min. | | |
| 0 | 0.61 | 0.41 | 00.7 |
| 0.5 | 732.7* | 64.41 | 4488.6* |
| 1.2 | ND | ND | ND |
| 4.0 | ND | ND | ND |

ND—not detectable
*half-life lasts longer than the experiment and is inferred from collected data.

Results, presented in Table 2 and FIGS. 2 and 3 show that improved chimeric DNases of present invention have enhanced DNase activity as compared with wild type bovine DNaseI enzyme and, depending on the origin of the fused ComEA-like domain, are capable to retain activity in increased salt concentrations. Even at 1 M NaCl wild type bovine DNaseI is essentially inactive and does not degrade DNA, while both fusion proteins retain DNase activity. Chimeric DNaseDT protein obtained using ComEA-like domain from hyperhalophile Thioalkalivibrio sp. K90mix exhibits detectable DNase activity even at such extreme conditions as 4 M NaCl, while chimeric DNaseBS protein shows enhanced DNaseI activity at moderate or low salt concentrations. The ComEA domain from B. subtilis is naturally exposed to the growth environment of the microorganism as it is located at the outer membrane and captures extracellular DNR. Bacillus subtilis is known to tolerate salinity fluctuations but basically favors low salt conditions. Therefore this domain should bind DNA at low or moderate ionic strength and should be optimal fusion partner for DNaseI at such conditions. Data presented in FIG. 2 shows that DNaseBS retains its activity at ionic strength up to 100 mM NaCl, while wild type DNaseI activity is already inhibited even by such low ionic strengths. Use of different ComEA-like domains as fusion partners with DNaseI according to present invention would thereby enable generation of a number of chimeric DNase proteins possessing enzymatic properties desirable for specific molecular biology technique and application, such as DNA degradation in high ionic strength buffers, or hyperactive DNase active in majority of commonly used molecular biology buffers.

In many cases, where DNA digestion is required, buffer does not contain extreme salt concentrations, however it is known that salt concentrations above 50-100 mM of a monavalent salt are inhibitory for bovine DNaseI. Equivalent ionic strengths are commonly found in many buffers commonly used molecular biology experimental workflow were DNaseI activity is required. Examples of such buffers could be reverse transcription buffers, in vitro transcription buffers, qPCR buffers. Therefore, it would be desirable to have a DNaseI variant, which would retain high activity at low to moderate salt concentrations. In order to examine this aspect we have performed monitored digestion of a fluorescently labeled DNA duplex. Resulting data presented in FIG. 3.

In FIG. 3, fluorescence curves for DNaseI and DNaseDT and DNaseBS corresponding to reactions performed in different salinity buffers along with quantified data are presented. For each enzyme the reference activity was the activity of a particular enzyme at 0 mM NaCl. Changes in activities due to increase in ionic strength up to 50 mM and 100 mM NaCl are given in FIG. 3 for the three enzymes. Therefore as we see, when NaCl concentration is increased up to 100 mM NaCl the activity of wild type DNaseI decreases by 78.2%. In the case of DNaseDT fusion situation is noticeably better—when ionic strength increases up to 100 mM NaCl, activity decreases by 68.5%. Therefore DNaseDT is more resistant to ionic strength (in the range of 0-100 mM NaCl) than wild type DNaseI. In the case of DNaseBS we see a more dramatic effect of the added domain: increase of NaCl concentration up to 50 mM NaCl increases measured activity up to 0.1% and increase of NaCl concentration up to 100 mM NaCl increases measured activity up to 0.08%. Therefore we may infer that activity of DNaseBS is essentially insensitive to ionic strength up to 100 mM NaCl. All in all both fusions (DNaseBS and DNaseDT) should be more efficient than wild type DNaseI in many molecular biology experimental workflows where DNA digestion is required and buffers contain salt.

### Example 3

A further logic step was to test these improved versions of DNaseI in real-life molecular biology workflow/applications; RNA purification and elimination of template DNA after transcription. Both applications require higher ionic strength, which is unfavorable for WT DNase.

### Example 3.1. Removal of Contaminating DNA During RNA Purification

In many RNA analysis techniques it is crucial to prepare RNA preparations free from contaminating DNA. As many buffers used in RNA purification workflow contain high amount of salts and wild type DNaseI is extremely ineffective in buffers of higher ionic strength, this makes RNA purification not an easy task. Effectiveness of improved DNases of present invention in digesting DNA during RNA purification was evaluated. Digestions were performed directly on the column filter using RNA obtained by using buffers of high ionic strength. We followed a modified protocol of GeneJET Whole Blood RNA Purification Mini Kit (Thermo Fisher Scientific) and used RNA purification columns supplied by manufacturer. During experiment we have purified total blood RNA. Four arbitrary blood samples were analyzed. DNA digestion was performed directly on a filter of a RNA purification column, were respective DNase enzyme was loaded on the filter together with 20 μl of reaction buffer. This was performed as an additional wash step. The following buffer was used for DNA digestion: 22.5 mM Tris—HCl, pH 7.5; 1,125 M NaCl; 10 mM MnCl2. Different amounts of respective DNase enzymes were used: 48 μM of DNaseI and 12 μM of DNaseBS and DNaseDT. As discussed above, 48 μM of DNaseI is molary equivalent to ˜40 Kunitz units of DNaseI and 12 μM is molary equivalent to ˜10 Kunitz units of DNaseI. For this assay only DNaseI, DNaseDT and DNaseBS preparations were used as they had no effect on RNA quality, while preparation of DNaseTA contained too much RNases (data not shown) and was eliminated from experiment.

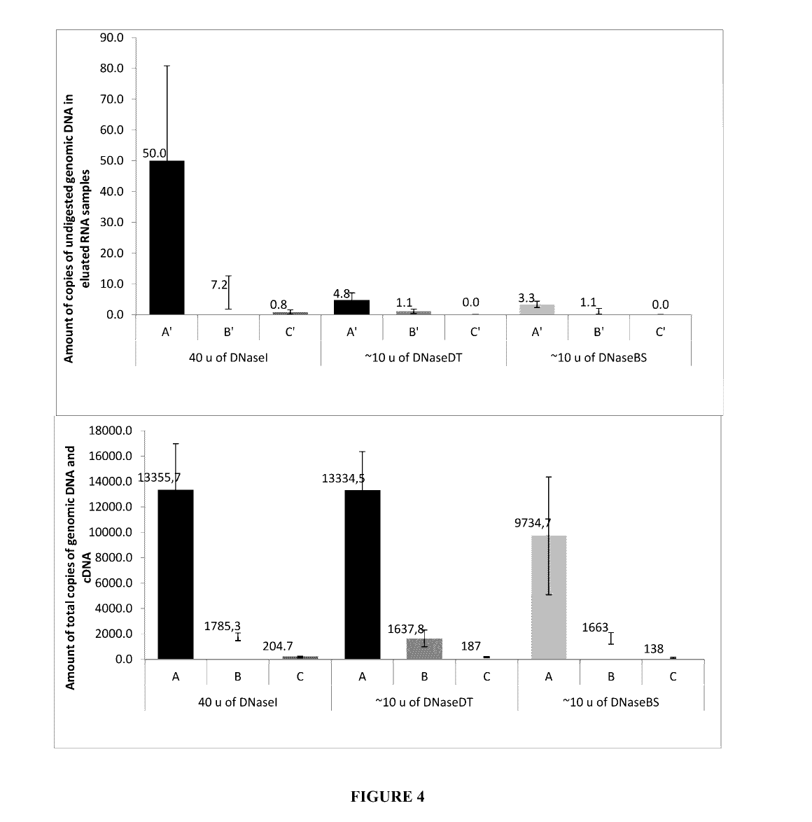

Eluted purified RNA was analyzed by RT-qPCR as 100× diluted, 10× diluted as well as undiluted samples. Results of RT-qPCR assays are presented in FIG. 4.

Results:

As it is shown in FIG. 4, 12 μM amount of both chimeric DNase proteins are sufficient to remove contaminating genomic DNA in about ten times more efficiently than 48 μM of DNaseI. As we see, when DNaseBS is used, ˜3 copies of genomic DNA are left. In case of DNaseDT only ˜5 copies are detected. Contrastingly, when wild type DNaseI is used, we have even ˜50 copies of undigested genomic DNA. Also, it is evident from data in FIG. 4 that detected RNA quantity is essentially similar in the cases of all three enzymes. Overall data in FIG. 4 indicates that chimeric DNases of present invention exhibit increased DNA digestion efficiency in buffers containing salts.

### Example 3.2. Removal of Template DNA after In Vitro Transcription Reaction

In many cases it is necessary to remove template DNA after in vitro transcription reactions. Often high yield transcription reactions are highly viscous due to extremely high concentration of transcribed RNA. Also reaction buffers for transcription have high quantities of magnesium ions and no calcium ions. Therefore transcription reaction buffers are unfavorable for wild type DNaseI.

Effectiveness of DNA digestion in transcription reactions was evaluated for wild type bovine DNaseI, DNaseTA and both chimeric enzymes, DNaseBS and DNaseDT, by performing high yield transcription reaction producing up to 50 μg RNA. Transcription reactions were performed using “TranscriptAid™ T7 High Yield Transcription Kit” (Thermo Fisher Scientific). As a template 1 μg control DNA from the kit was used. After transcription reaction (2 h, 37° C. temp.), undiluted and 5× diluted samples were treated with varying amounts of respective DNase enzymes: 1.2, 2.4, 12 μM. It was previously determined (data not shown) that 1 Kuntiz unit of bovine DNaseI corresponds to ˜1.2 μM, therefore we treated samples with such amounts of enzymes, which would be molary equivalent to 1, 2, 10 Kunitz units of bovine DNaseI.

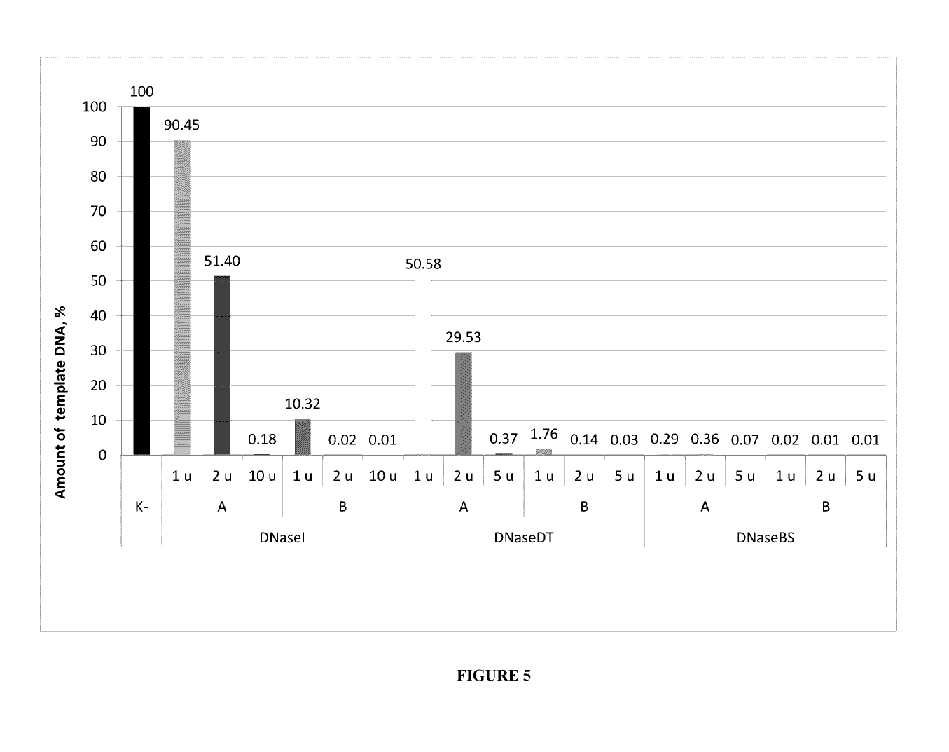

Dilutions were performed in the following buffer: 10 mM Tris—HCl, pH 7.5; 0.1 mM CaCl2. A control sample was a sample, which was not treated with any DNase. Digestion reactions were performed 15′ at 37° C. and stopped by addition of 2 μl 0.5 M EDTA and heating 10′ at 65° C. Before analysis by qPCR, samples were diluted with nuclease free water (Thermo Fisher Scientific) up to 1.8×10⁷ copies/μl in the control samples. 2 μl of diluted samples were used for qPCR reactions. Results are presented in FIG. 5.

Results:

As shown in FIG. 5, wild type bovine DNaseI performs poorly in the undiluted samples as even 90% of DNA remains undigested using 1.2 μM of the enzyme. Increased concentration of DNaseI results in increased percentage of digested DNA: 2.4 μM of enzyme digests about half of template DNA, while use of 12 μM concentration results in 0.2% of undigested template. As seen in 5× diluted sample, after digestion with 2.4 μM of wild type DNaseI only 0.02% of template remains intact.

Secondly, it is clear that halophylic DNaseTA performs in this application not much better than wild type DNaseI: after in vitro transcription reaction even 12 μM concentration of this enzyme (molary equivalent to 10 units of bovine DNaseI) in a 5× diluted sample leave more than 10% of total DNA.

Thirdly, it is obvious that both chimeric DNase enzymes, DNaseDT and DNaseBS, are superior as compared to DNaseI in this application. Use of only 1.2 μM of DNaseBS in even undiluted sample resulted in only 0.02% remaining undigested template DNA, which is 300 times less than in case of wild type DNaseI. In case of DNaseDT enzyme we can see that certain enhancements over wild type DNaseI activity are noticeable in this application, although quite minute: generally this enzymes efficiency is double as compared to that of wild type DNaseI in transcription reaction buffer

Results presented in FIGS. 4 and 5 show that chimeric DNases of present invention are superior to wild type DNaseI in at least two applications: While template removal immediately after in vitro transcription reaction is performed at moderate ionic strength, RNA purification requires DNase to be active at quite high ionic strength. In the RNA purification example (FIG. 4) both chimeric fusions performed much better than the wild type bovine DNaseI. In the case of post transcription in vitro template elimination both fusions outperformed wild type DNaseI as well.

From the examples described herein, one skilled in the art can easily ascertain the essential principles of this invention and without departing from the spirit and scope thereof, can make various modifications and changes of the invention in adapting to specific uses and conditions.

1. A deoxyribonuclease comprising: (a) an amino acid sequence having at least 85% sequence identity with the sequence of a eukaryotic DNase I; and (b) an amino acid sequence capable of binding nucleic acid non-specifically comprising at least one helix-hairpin-helix motif.

2. A deoxyribonuclease according to claim 1 wherein the eukaryotic DNaseI is bovine DNase I.

3. A deoxyribonuclease according to claim 1 or claim 2 wherein the eukaryotic DNase I has an SEQ ID NO: 2 or an amino acid sequence having at least 85% identity with SEQ ID NO: 2.

4. A deoxyribonuclease according to any preceding claim wherein the amino acid sequence capable of binding nucleic acid non-specifically comprises two helix-hairpin-helix motifs.

5. A deoxyribonuclease according to any preceding claim wherein the amino acid sequence of the at least one helix-hairpin-helix motif is a ComEA protein helix-hairpin-helix sequence.

6. A deoxyribonuclease according to claim 5 wherein the ComEA protein helix-hairpin-helix sequence is from an organism of a genus selected from Bacillus, Thioalkalivibrio or Halomonas.

7. A deoxyribonuclease according to any preceding claim wherein the amino acid sequence capable of binding nucleic acid non-specifically consists of:
(i) a sequence having at least 85% sequence identity with SEQ ID NO: 15, wherein the sequence having at least 85% sequence identity with SEQ ID NO: 15 comprises SEQ ID NO: 31 and SEQ ID NO: 32;
(ii) a sequence having at least 85% sequence identity with SEQ ID NO: 22, wherein the sequence having at least 85% sequence identity with SEQ ID NO: 22 comprises SEQ ID NO: 29 and SEQ ID NO: 30; or
(iii) a sequence having at least 85% sequence identity with SEQ ID NO: 37, wherein the sequence having at least 85% sequence identity with SEQ ID NO: 37 comprises SEQ ID NO: 38 and SEQ ID NO: 39

8. A deoxyribonuclease according to any preceding claim wherein the amino acid sequence capable of binding nucleic acid non-specifically comprises SEQ ID NO: 33, SEQ ID NO: 34 or SEQ ID NO: 40.

9. A deoxyribonuclease according to any preceding claim which has an activity at 50 Mm NaCl and/or at 100 mM NaCl greater than that of a wild-type bovine DNase I having SEQ ID NO: 2.

10. A deoxyribonuclease according to any preceding claim which has a higher affinity to double-stranded and/or single-stranded DNA compared to a wild-type bovine DNaseI having SEQ ID NO: 2.

11. A deoxyribonuclease having SEQ ID NO: 24 or SEQ ID NO: 26.

12. A polynucleotide encoding the deoxyribonuclease according to any one of claims 1 to 11.

13. A polynucleotide according to claim 12 comprising SEQ ID NO: 23 or 25 or a sequence having at least 85% sequence identity thereto.

14. A vector comprising the polynucleotide of claim 12 or claim 13.

15. A host cell which comprises the vector of claim 14 or the polynucleotide of claim 12 or claim 13.

16. A method of producing the deoxyribonuclease of any one of claims 1 to 11 comprising the steps of culturing the host cell of claim 15 under conditions which allow for the expression of the deoxyribonuclease.

17. A composition comprising a deoxyribonuclease of any one of claims 1 to 11 and a buffer.

18. A composition according to claim 17, wherein the buffer comprises at least one of TrisHCl, CaCl₂, MgCl₂ and glycerol.

19. A kit for removing DNA from a sample comprising a deoxyribonuclease according to any one of claims 1 to 11 and a reaction buffer.

20. Use of a deoxyribonuclease according to any one of claims 1 to 11 or a kit according to claim 19 to digest DNA in a sample.

21. Use according to claim 20 wherein the sample comprises RNA.

22. A method for removing DNA from a sample comprising contacting the sample with the deoxyribonuclease of any one of claims 1 to 11 under conditions that allow the deoxyribonuclese to digest the DNA.

23. A method according to claim 22 wherein the conditions include from 50 mM to 4 M NaCl.

24. A method according to claim 22 or claim 23 wherein the sample comprises RNA.


SEQUENCE LISTING (44 sequences)

SEQ ID NO: 1 - DNA, 873 bp, Bos taurus
atggcgggttctcatcatcatcatcatcatggtatggctagcatgactggtggacagcaaatgggtcgctccggtgatgatgatgacaagctgaagatagcagccttcaacatccgcacctttggggagaccaagatgtccaatgctacgctcgccagctacattgttcggatcgtgcgtcgttacgacatcgtcctcatccaggaggtcagagacagccacctggtggctgtggggaagctcctggactatctcaaccaggatgacccaaacacctaccactatgtggtcagtgagccgctgggccgcaacagctacaaggagcgctacctctttctgttcagacccaacaaggtgtccgtgctggacacctaccagtacgacgacggctgcgagtcctgcgggaacgacagcttcagccgggagcccgctgtggtcaagttctcatcccactccaccaaggtcaaggaatttgccattgttgccctgcactcggccccatcggacgcagtggctgagattaattctctctacgatgtctacctggatgtccagcagaagtggcacttgaacgatgtcatgttgatgggcgatttcaatgctgactgcagctacgtgacctcctcgcagtggtcatccatccgcctgcgtacgagctccaccttccagtggctgattcctgacagtgccgacaccacggctacgtccacgaactgcgcctatgacaggatcgtggtcgcagggtctctgctccagagttctgtggttcctggctcggccgctccctttgacttccaagctgcatacggactgagcaatgagatggccctggccatcagtgaccattacccggtggaggtgacgctgacataa

SEQ ID NO: 2 - protein, 290 aa, Bos taurus
MAGSHHHHHHGMASMTGGQQMGRSGDDDDKLKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLT

SEQ ID NO: 3 - DNA, 1038 bp, Thioalkalivibrio sp. (strain K90mix)
atgctgcgcctggcaagctggaacatccagcacctgggctggaacgtcggtaaggactacccggccgtcgcacgcatcgcggcgcagttcgactttctggcgatccaagaagtcatgaacgccgagggtatttaccgcctgcgtgacaccctggaagacgcgaccggtgccgagtggtccgttctgtactccgacgcgctgggccgcaacacctatcgtgaaaagtacgcgttcctgtggcgtgaggccgcagttgagtatgtcggtggtgcgctgacctacatcgacgaggccgaccgttttgcccgtgagccgttctccgcggtctttcgttcccgtggcacggatcagcatttcctggccgccacggttcacatcacctacggcgaccgtgttgcggatcgtgtcgaggagatcgaggcactgcgtcgttactgggactggctggcggacgtcatgccggagtacgccggcgaacgcatcctgttcggcgacttcaatctgccgccgcaccacgacggttgggcctcgatgcgtgcggttgccgaaccgctggtcaccgagggcgccaccaccctgtccacgcatgaccgtcgctacgcgaatctgtacgacaatctgtgggttccgaaggatcacacgctgccgctgggtgatgccggcatcctgccgttcccggtcgttctgagtgaggtaaccggtgtctactgggatcacgaaaaggcccgcgaccgtgtctccgaccatgccccggtttatgtactgttcgaaggtaacacgctgcacgacgcggttgtcgcggagatcgccgatcaagaagcgggctgcatcgatctgaatcgcgcctcggtaagcgaactgaccgccctgccgcatattggcgaggcacgtgcggaggcgatcaaggacggtcgtccgtggaatgccgttcgtgacctgaaagagattcgcggtatcggtgctggtcgtctggaggagatcaaggcgcgtggtgaggcctgcatcgagccgggccatcaccatcaccaccacggctaa

SEQ ID NO: 4 - protein, 345 aa, Thioalkalivibrio sp. (strain K90mix)
MLRLASWNIQHLGWNVGKDYPAVARIAAQFDFLAIQEVMNAEGIYRLRDTLEDATGAEWSVLYSDALGRNTYREKYAFLWREAAVEYVGGALTYIDEADRFAREPFSAVFRSRGTDQHFLAATVHITYGDRVADRVEEIEALRRYWDWLADVMPEYAGERILFGDFNLPPHHDGWASMRAVAEPLVTEGATTLSTHDRRYANLYDNLWVPKDHTLPLGDAGILPFPVVLSEVTGVYWDHEKARDRVSDHAPVYVLFEGNTLHDAVVAEIADQEAGCIDLNRASVSELTALPHIGEARAEAIKDGRPWNAVRDLKEIRGIGAGRLEEIKARGEACIEPGHHHHHHG

SEQ ID NO: 5 - DNA, 26 bp, Artificial Sequence
  Primer
gccgccacggttgcaatcacctacgg

SEQ ID NO: 6 - DNA, 41 bp, Artificial Sequence
  Primer
gtggtggtgatggtgatggcccggctcgatgcaggcctcac

SEQ ID NO: 7 - DNA, 37 bp, Artificial Sequence
  Primer
agaaggagatataactatgctgcgcctggcaagctgg

SEQ ID NO: 8 - DNA, 1038 bp, Artificial Sequence
  Coding sequence of DNaseTA H134A mutant
atgctgcgcctggcaagctggaacatccagcacctgggctggaacgtcggtaaggactacccggccgtcgcacgcatcgcggcgcagttcgactttctggcgatccaagaagtcatgaacgccgagggtatttaccgcctgcgtgacaccctggaagacgcgaccggtgccgagtggtccgttctgtactccgacgcgctgggccgcaacacctatcgtgaaaagtacgcgttcctgtggcgtgaggccgcagttgagtatgtcggtggtgcgctgacctacatcgacgaggccgaccgttttgcccgtgagccgttctccgcggtctttcgttcccgtggcacggatcagcatttcctggccgccacggttgcaatcacctacggcgaccgtgttgcggatcgtgtcgaggagatcgaggcactgcgtcgttactgggactggctggcggacgtcatgccggagtacgccggcgaacgcatcctgttcggcgacttcaatctgccgccgcaccacgacggttgggcctcgatgcgtgcggttgccgaaccgctggtcaccgagggcgccaccaccctgtccacgcatgaccgtcgctacgcgaatctgtacgacaatctgtgggttccgaaggatcacacgctgccgctgggtgatgccggcatcctgccgttcccggtcgttctgagtgaggtaaccggtgtctactgggatcacgaaaaggcccgcgaccgtgtctccgaccatgccccggtttatgtactgttcgaaggtaacacgctgcacgacgcggttgtcgcggagatcgccgatcaagaagcgggctgcatcgatctgaatcgcgcctcggtaagcgaactgaccgccctgccgcatattggcgaggcacgtgcggaggcgatcaaggacggtcgtccgtggaatgccgttcgtgacctgaaagagattcgcggtatcggtgctggtcgtctggaggagatcaaggcgcgtggtgaggcctgcatcgagccgggccatcaccatcaccaccacggctaa

SEQ ID NO: 9 - protein, 345 aa, Artificial Sequence
  Amino acid sequence of DNaseTA H134A mutant
MLRLASWNIQHLGWNVGKDYPAVARIAAQFDFLAIQEVMNAEGIYRLRDTLEDATGAEWSVLYSDALGRNTYREKYAFLWREAAVEYVGGALTYIDEADRFAREPFSAVFRSRGTDQHFLAATVAITYGDRVADRVEEIEALRRYWDWLADVMPEYAGERILFGDFNLPPHHDGWASMRAVAEPLVTEGATTLSTHDRRYANLYDNLWVPKDHTLPLGDAGILPFPVVLSEVTGVYWDHEKARDRVSDHAPVYVLFEGNTLHDAVVAEIADQEAGCIDLNRASVSELTALPHIGEARAEAIKDGRPWNAVRDLKEIRGIGAGRLEEIKARGEACIEPGHHHHHHG

SEQ ID NO: 10 - DNA, 45 bp, Artificial Sequence
  Primer
gtggtggtgatggtgatggccaccttcgaacagtacataaaccgg

SEQ ID NO: 11 - DNA, 34 bp, Artificial Sequence
  Primer
agaaggagatataactatgctgcgcctggcaagc

SEQ ID NO: 12 - DNA, 801 bp, Artificial Sequence
  Coding sequence of DNaseTA deletion mutant
atgctgcgcctggcaagctggaacatccagcacctgggctggaacgtcggtaaggactacccggccgtcgcacgcatcgcggcgcagttcgactttctggcgatccaagaagtcatgaacgccgagggtatttaccgcctgcgtgacaccctggaagacgcgaccggtgccgagtggtccgttctgtactccgacgcgctgggccgcaacacctatcgtgaaaagtacgcgttcctgtggcgtgaggccgcagttgagtatgtcggtggtgcgctgacctacatcgacgaggccgaccgttttgcccgtgagccgttctccgcggtctttcgttcccgtggcacggatcagcatttcctggccgccacggttcacatcacctacggcgaccgtgttgcggatcgtgtcgaggagatcgaggcactgcgtcgttactgggactggctggcggacgtcatgccggagtacgccggcgaacgcatcctgttcggcgacttcaatctgccgccgcaccacgacggttgggcctcgatgcgtgcggttgccgaaccgctggtcaccgagggcgccaccaccctgtccacgcatgaccgtcgctacgcgaatctgtacgacaatctgtgggttccgaaggatcacacgctgccgctgggtgatgccggcatcctgccgttcccggtcgttctgagtgaggtaaccggtgtctactgggatcacgaaaaggcccgcgaccgtgtctccgaccatgccccggtttatgtactgttcgaaggtggccatcaccatcaccaccacggctaa

SEQ ID NO: 13 - protein, 266 aa, Artificial Sequence
  Amino acid sequence of DNaseTA deletion mutant
MLRLASWNIQHLGWNVGKDYPAVARIAAQFDFLAIQEVMNAEGIYRLRDTLEDATGAEWSVLYSDALGRNTYREKYAFLWREAAVEYVGGALTYIDEADRFAREPFSAVFRSRGTDQHFLAATVHITYGDRVADRVEEIEALRRYWDWLADVMPEYAGERILFGDFNLPPHHDGWASMRAVAEPLVTEGATTLSTHDRRYANLYDNLWVPKDHTLPLGDAGILPFPVVLSEVTGVYWDHEKARDRVSDHAPVYVLFEGGHHHHHHG

SEQ ID NO: 14 - DNA, 237 bp, Thioalkalivibrio sp. (strain K90mix)
ggtaacacgctgcacgacgcggttgtcgcggagatcgccgatcaagaagcgggctgcatcgatctgaatcgcgcctcggtaagcgaactgaccgccctgccgcatattggcgaggcacgtgcggaggcgatcaaggacggtcgtccgtggaatgccgttcgtgacctgaaagagattcgcggtatcggtgctggtcgtctggaggagatcaaggcgcgtggtgaggcctgcatcgag

SEQ ID NO: 15 - protein, 79 aa, Thioalkalivibrio sp. (strain K90mix)
GNTLHDAVVAEIADQEAGCIDLNRASVSELTALPHIGEARAEAIKDGRPWNAVRDLKEIRGIGAGRLEEIKARGEACIE

SEQ ID NO: 16 - DNA, 40 bp, Artificial Sequence
  Primer
cggtggaggtgacgctgacaggtaacacgctgcacgacgc

SEQ ID NO: 17 - DNA, 38 bp, Artificial Sequence
  Primer
ggagatgggaagtcattactcgatgcaggcctcaccac

SEQ ID NO: 18 - DNA, 44 bp, Artificial Sequence
  Primer
ggtgatgatgatgacaagctgaagatagcagccttcaacatccg

SEQ ID NO: 19 - DNA, 42 bp, Artificial Sequence
  Primer
cggtggaggtgacgctgacaggagaggaaacagcagtgcagc

SEQ ID NO: 20 - DNA, 49 bp, Artificial Sequence
  Primer
ggagatgggaagtcattactttactgtaatggaagactttattttctca

SEQ ID NO: 21 - DNA, 255 bp, Bacillus subtilis (strain 168)
ggagaggaaacagcagtgcagcaaggtggcggagggtctgtccaaagcgatggagggaagggagcgctggtgaatatcaatacagcaaccttagaggagttacaaggcatctcaggggtggggccatccaaagctgaagctattattgcataccgggaggaaaacggtcgtttccaaacaattgaagatatcactaaggtttcaggaataggtgaaaagtcatttgagaaaataaagtcttccattacagtaaag

SEQ ID NO: 22 - protein, 85 aa, Bacillus subtilis (strain 168)
GEETAVQQGGGGSVQSDGGKGALVNINTATLEELQGISGVGPSKAEAIIAYREENGRFQTIEDITKVSGIGEKSFEKIKSSITVK

SEQ ID NO: 23 - DNA, 1128 bp, Artificial Sequence
  Coding sequence of DNaseBS
atggcgggttctcatcatcatcatcatcatggtatggctagcatgactggtggacagcaaatgggtcgctccggtgatgatgatgacaagctgaagatagcagccttcaacatccgcacctttggggagaccaagatgtccaatgctacgctcgccagctacattgttcggatcgtgcgtcgttacgacatcgtcctcatccaggaggtcagagacagccacctggtggctgtggggaagctcctggactatctcaaccaggatgacccaaacacctaccactatgtggtcagtgagccgctgggccgcaacagctacaaggagcgctacctctttctgttcagacccaacaaggtgtccgtgctggacacctaccagtacgacgacggctgcgagtcctgcgggaacgacagcttcagccgggagcccgctgtggtcaagttctcatcccactccaccaaggtcaaggaatttgccattgttgccctgcactcggccccatcggacgcagtggctgagattaattctctctacgatgtctacctggatgtccagcagaagtggcacttgaacgatgtcatgttgatgggcgatttcaatgctgactgcagctacgtgacctcctcgcagtggtcatccatccgcctgcgtacgagctccaccttccagtggctgattcctgacagtgccgacaccacggctacgtccacgaactgcgcctatgacaggatcgtggtcgcagggtctctgctccagagttctgtggttcctggctcggccgctccctttgacttccaagctgcatacggactgagcaatgagatggccctggccatcagtgaccattacccggtggaggtgacgctgacaggagaggaaacagcagtgcagcaaggtggcggagggtctgtccaaagcgatggagggaagggagcgctggtgaatatcaatacagcaaccttagaggagttacaaggcatctcaggggtggggccatccaaagctgaagctattattgcataccgggaggaaaacggtcgtttccaaacaattgaagatatcactaaggtttcaggaataggtgaaaagtcatttgagaaaataaagtcttccattacagtaaagtaa

SEQ ID NO: 24 - protein, 375 aa, Artificial Sequence
  Amino acid sequence of DNaseBS
MAGSHHHHHHGMASMTGGQQMGRSGDDDDKLKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLTGEETAVQQGGGGSVQSDGGKGALVNINTATLEELQGISGVGPSKAEAIIAYREENGRFQTIEDITKVSGIGEKSFEKIKSSITVK

SEQ ID NO: 25 - DNA, 1110 bp, Artificial Sequence
  Coding sequence of DNaseDT
atggcgggttctcatcatcatcatcatcatggtatggctagcatgactggtggacagcaaatgggtcgctccggtgatgatgatgacaagctgaagatagcagccttcaacatccgcacctttggggagaccaagatgtccaatgctacgctcgccagctacattgttcggatcgtgcgtcgttacgacatcgtcctcatccaggaggtcagagacagccacctggtggctgtggggaagctcctggactatctcaaccaggatgacccaaacacctaccactatgtggtcagtgagccgctgggccgcaacagctacaaggagcgctacctctttctgttcagacccaacaaggtgtccgtgctggacacctaccagtacgacgacggctgcgagtcctgcgggaacgacagcttcagccgggagcccgctgtggtcaagttctcatcccactccaccaaggtcaaggaatttgccattgttgccctgcactcggccccatcggacgcagtggctgagattaattctctctacgatgtctacctggatgtccagcagaagtggcacttgaacgatgtcatgttgatgggcgatttcaatgctgactgcagctacgtgacctcctcgcagtggtcatccatccgcctgcgtacgagctccaccttccagtggctgattcctgacagtgccgacaccacggctacgtccacgaactgcgcctatgacaggatcgtggtcgcagggtctctgctccagagttctgtggttcctggctcggccgctccctttgacttccaagctgcatacggactgagcaatgagatggccctggccatcagtgaccattacccggtggaggtgacgctgacaggtaacacgctgcacgacgcggttgtcgcggagatcgccgatcaagaagcgggctgcatcgatctgaatcgcgcctcggtaagcgaactgaccgccctgccgcatattggcgaggcacgtgcggaggcgatcaaggacggtcgtccgtggaatgccgttcgtgacctgaaagagattcgcggtatcggtgctggtcgtctggaggagatcaaggcgcgtggtgaggcctgcatcgagtaa

SEQ ID NO: 26 - protein, 369 aa, Artificial Sequence
  Amino acid sequence of DNaseDT
MAGSHHHHHHGMASMTGGQQMGRSGDDDDKLKIAAFNIRTFGETKMSNATLASYIVRIVRRYDIVLIQEVRDSHLVAVGKLLDYLNQDDPNTYHYVVSEPLGRNSYKERYLFLFRPNKVSVLDTYQYDDGCESCGNDSFSREPAVVKFSSHSTKVKEFAIVALHSAPSDAVAEINSLYDVYLDVQQKWHLNDVMLMGDFNADCSYVTSSQWSSIRLRTSSTFQWLIPDSADTTATSTNCAYDRIVVAGSLLQSSVVPGSAAPFDFQAAYGLSNEMALAISDHYPVEVTLTGNTLHDAVVAEIADQEAGCIDLNRASVSELTALPHIGEARAEAIKDGRPWNAVRDLKEIRGIGAGRLEEIKARGEACIE

SEQ ID NO: 27 - protein, 30 aa, Artificial Sequence
  Oligonucleotide
GTTGGTGGGTTTGGGTGTGGGTTTGTGTTT

SEQ ID NO: 28 - protein, 30 aa, Artificial Sequence
  Oligonucleotide
AAACACAAACCCACACCCAAACCCACCAAC

SEQ ID NO: 29 - protein, 20 aa, Bacillus subtillis
EELQGISGVGPSKAEAIIAY

SEQ ID NO: 30 - protein, 20 aa, Bacillus subtilis
EDITKVSGIGEKSFEKIKSS

SEQ ID NO: 31 - protein, 20 aa, Thioalkalivibrio sp. (strain K90mix)
SELTALPHIGEARAEAIKDG

SEQ ID NO: 32 - protein, 20 aa, Thioalkalivibrio sp. (strain K90mix)
RDLKEIRGIGAGRLEEIKAR

SEQ ID NO: 33 - protein, 50 aa, Bacillus subtilis
EELQGISGVGPSKAEAIIAYREENGRFQTIEDITKVSGIGEKSFEKIKSS

SEQ ID NO: 34 - protein, 46 aa, Thioalkalivibrio sp. (strain K90mix)
SELTALPHIGEARAEAIKDGRPWNAVRDLKEIRGIGAGRLEEIKAR

SEQ ID NO: 35 - protein, 81 aa, Bacillus subtilis
GEETAVQQGGGGSVQSDGGKGALVNINTATLEELQGISGVGPSKAEAIIAYREENGRFQTIEDITKVSGIGEKSFEKIKSS

SEQ ID NO: 36 - protein, 73 aa, Thioalkalivibrio sp. (strain K90mix)
GNTLHDAVVAEIADQEAGCIDLNRASVSELTALPHIGEARAEAIKDGRPWNAVRDLKEIRGIGAGRLEEIKAR

SEQ ID NO: 37 - protein, 82 aa, Halomonas sp. TD01
TAELLLSSFVGGAYEAANDPIYCIDLNEATAEQLTELPNVGPVRAEQIIEMRPWTNADELSQVSGLGVASVETIKNSGLVCP

SEQ ID NO: 38 - protein, 20 aa, Halomonas sp. TD01
EQLTELPNVGPVRAEQIIEM

SEQ ID NO: 39 - protein, 20 aa, Halomonas sp. TD01
DELSQVSGLGVASVETIKNS

SEQ ID NO: 40 - protein, 46 aa, Halomonas sp. TD01
EQLTELPNVGPVRAEQIIEMRPWTNADELSQVSGLGVASVETIKNS

SEQ ID NO: 41 - protein, 77 aa, Halomonas sp. TD01
TAELLLSSFVGGAYEAANDPIYCIDLNEATAEQLTELPNVGPVRAEQIIEMRPWTNADELSQVSGLGVASVETIKNS

SEQ ID NO: 42 - protein, 31 aa, Bacillus subtilis
GEETAVQQGGGGSVQSDGGKGALVNINTATL

SEQ ID NO: 43 - protein, 27 aa, Thioalkalivibrio sp. (strain K90mix)
GNTLHDAVVAEIADQEAGCIDLNRASV

SEQ ID NO: 44 - protein, 31 aa, Halomonas sp. TD01
TAELLLSSFVGGAYEAANDPIYCIDLNEATA